(19) United States
(12) Patent Application Publication
(10) Pub. No.: US 2023/0265378 A1
(43) Pub. Date: Aug. 24, 2023

(54) STIMULATING CELLULAR PRODUCTION OF EXOSOMES

(71) Applicant: ROYAL MELBOURNE INSTITUTE OF TECHNOLOGY, Melbourne, Victoria (AU)

(72) Inventors: Lizebona August AMBATTU, Melbourne (AU); Shwathy RAMESAN, Melbourne (AU); Leslie YEO, Melbourne (AU)

(21) Appl. No.: 18/041,238

(22) PCT Filed: Aug. 10, 2021
(86) PCT No.: PCT/AU2021/050876

§ 371 Date: Feb. 10, 2023

(30) Foreign Application Priority Data
Aug. 11, 2020 (AU) 2020902838

(51) Int. Cl. C12M 1/42; C12M 1/32; A61K 9/50; C12N 5/09

(52) U.S. Cl. CPC C12M 35/04; C12M 23/12; A61K 9/5068; C12N 5/0693; C12N 2521/10

(57) ABSTRACT

Embodiments relate to a method and apparatus for stimulating the production of extracellular vesicles (EVs) from a population of cells. Some embodiments relate to a method for stimulating the production of extracellular vesicles (EVs) from a population of cells, the method comprising: (i) exposing a culture media comprising the population of cells to acoustic wave energy and (ii) harvesting the EVs produced from the population of cells following the exposure. Some embodiments relate to an apparatus for use in stimulating the production of EVs from a population of cells, the apparatus comprising: (i) an acoustic wave generator configured to generate acoustic energy at a selected power and frequency; and (ii) a receptacle for accommodating a population of cells in a culture media, the receptacle being configured to receive acoustic energy generated by the acoustic wave generator.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### FIELD OF THE DISCLOSURE

Embodiments generally relate to methods and associated apparatus for stimulating the production of extracellular vesicles (EVs) from a population of cells.

### BACKGROUND

Exosomes are extracellular vesicles (EVs) between 30 and 150 nm in diameter that are secreted by all eukaryotic cells into the extracellular microenvironment. Unlike other subclasses of extracellular vesicles, they are released from multivesicular bodies rather than directly from the plasma membrane. Exosomes play an important role in intercellular communication by facilitating the transmission of macromolecules such as mRNA, miRNA, DNA, lipids and proteins between cells, and therefore their influence have been implicated in disease development and transmission. As such, there has been significant interest in their isolation from circulatory samples for disease detection, particularly cancer. Exosome-based liquid biopsies, for example, allow real-time profiling of a patient's tumour activity by isolating DNA or RNA from the exosome for further analysis, thus facilitating monitoring of disease progression without requiring invasive surgical procedures.

Being the facsimile of the cell, exosomes are also superior drug delivery vectors compared to synthetic polymers or viruses since their lipid bilayer structure is similar to that of cell membranes, and are therefore considerably less likely to invoke an immune response. Besides their ability to traverse the blood-brain barrier these azoic entities are, in addition, known to induce transcriptomic and phenotypic changes and therefore play critical roles in stem cell differentiation and modulating the tumour niche. Moreover, as all eukaryotic cells produce exosomes and internalise them, they are able to target any cell type, and have recently been used for therapeutic targeting of the oncogene KRAS, considered among the most challenging of drug targets. Consequently, there are currently widespread efforts to harness them as carriers in gene and protein therapy.

A significant technical challenge in practice, however, lies in obtaining adequate quantities of pure exosomes that are sufficiently homogeneous through cell culture (over several days) and subsequently isolating them. A number of methods to enhance exosome yield have therefore been proposed. These, for example, involve chemical (e.g., ionomycin or intracellular calcium), biochemical (e.g., extracellular DNA or liposomes or altering proteomic content like introducing p5338), pH or mechanical (e.g., cyclic stretching) stimuli; methods to induce cell hypoxia; cytoskeletal protein alteration; gene overexpression; or exposure to thermal, oxidative, photodynamic or radiative stress. More recently, a cell nanoporation technique that can be scaled for high throughput has also been demonstrated.

There are nevertheless a number of potential disadvantages to some of the aforementioned methods. The use of additives such as ionomycin and calcium phosphate, for example, while capable of enhancing exosome yield by two-and-one-half-fold within 2 to 72 hrs (approximately 0.03-1 fold/hr), are however dose dependent and overexposure of the cells to these chemicals can lead to a considerable reduction in their viability; similarly, exposing cells to ionising radiation can result in cell apoptosis. Thermal and oxidative stresses, on the other hand, have been reported to increase exosome yield by approximately 20- to 30-fold in 24 hrs (approximately 0.8-1.25 fold/hr) but can generate immunoresponsive exosomes which could impair their diagnostic or therapeutic potential. Given the role of heat shock responses in the exosome production mechanism in the cell nanoporation technique, a corollary to the enhancement afforded by the technique is the upregulation in p53 tumour suppressor protein activity, which can potentially result in undesirable development of a pro-invasive microenvironment. In any case, besides addressing low exosome yield, few of these methods, if any, are also able to circumvent lipidome and proteome heterogeneity in the exosome population, which constitutes a further barrier to translation of exosome therapies into clinical practice.

Accordingly, there is a need in the art for methods that produce exosomes in high yield that overcome the disadvantages of the prior art methods.

### SUMMARY OF THE DISCLOSURE

The present inventors have shown herein that an increase in exosome production in mammalian cells can be obtained by exposing the cells to acoustic stimulation while maintaining cell viability and proliferation. Moreover, the inventors have shown that repeated cycles of stimulation and recovery provides increased exosome production whilst avoiding proteome and lipidome heterogeneity in the exosome population, which is problematic for exosome therapeutics.

In a first aspect, the present disclosure provides a method for stimulating the production of extracellular vesicles (EVs) from a population of cells, the method comprising:

(i) exposing a culture media comprising the population of cells to acoustic wave energy and

(ii) harvesting the EVs produced from the population of cells following the exposure.

In one example, the method involves harvesting EVs which are secreted from the cells following the acoustic exposure.

In one example, the EVs are membrane vesicles. In a particular example, the EVs comprise a membrane bilayer, more preferably a cellular membrane bilayer. The EVs may comprise a homogenous or heterogeneous population of vesicles. For example, the EVs may be selected from exosomes or microvesicles (ectosomes), oncosomes or combinations thereof.

The term “exosome” as used herein is also intended to include dexosomes and texosomes.

In one example, the EVs have a size between about 10 nm and 1000 nm.

In one example, the EVs comprise greater than 80% exosomes, such as at least 85%, at least 90%, at least 92%, at least 95% or at least 98% or 100% exosomes.

In one example, the exosomes have a hydrodynamic diameter size of between 30-150 nm. In some examples, the exosomes have a size between 50-90 nm, or between 60-80 nm in diameter.

The EVs according to the disclosure are non-synthetic vesicles comprising a bilayer lipid membrane that surrounds an aqueous core. In some examples, the exosomes are devoid of nucleic acids. In another example, the exosomes comprise at least one species of nucleic acid, for example miRNA.

The method of the disclosure may further comprise a step of preparing a population of cells. The skilled person would appreciate that such a population of cells are exosome producing cells. The population of cells may be a homogenous or heterogeneous population of cells. For example, the population of cells may comprise at least 50%, at least 60%, at least 70%, at least 80%, at least 90%, at least 92%, at least 95% or at least 98% or 100% cells of homogenous type.

In one example, the population of cells are mammalian cells, such as human cells, or are derived from a mammalian tissue source. In one example, the cell population is an immune cell such as a T or B lymphocyte, a dendritic cell, a glioma, platelet, reticulocyte, neuron, intestinal epithelial cell or a tumour cell. In one example, the population of cells are human glioblastoma cells. In another example, the population of cells are adenocarcinoma human alveolar basal epithelial cells. In some examples, the population of cells are first cultured in an appropriate cell culture medium, for example in a culture flask and then re-seeding into smaller culture plate. In some examples, the cells are first cultured in a flask, e.g. T25 flask until reaching desired confluency, typically around 80-90%.

It will be understood that the EVs, more preferably the exosomes harvested from the population of cells will be derived from the said population of cells. By “derived from” it is meant that the exosome is naturally produced from its corresponding parent/source cell line. For example, A549 alveolar basal epithelial cells produce exosomes naturally and these exosomes are referred to herein as A549 exosomes to indicate source.

In certain examples, the population of cells are cultured in an appropriate cell culture media. Persons skilled in the art will be familiar with appropriate cell culture media for growing mammalian cells. Examples include Roswell Park Memorial Institute (RPMI) 1640 medium or Dulbecco's Modified Eagle Medium (D-MEM). The media may be supplemented with serum, antibiotics etc. as appropriate.

In certain examples, the cells and media are cultured in a suitable receptacle or reservoir. Examples, of suitable receptacles/reservoirs include tissue culture plates. In one example, the receptacle is a 2-well plate, a 4-well plate, an 8-well plate, a 16-well plate or higher. The cells may be plated at a density deemed appropriate by the skilled person. For example for an 8-well plate, the cells may be plated/seeded at a density of about 2×10⁵ cells/well to about 5×10⁵ cells/well, more preferably about 3-3.5×10⁵ cells/well. Prior to exposure to the acoustic wave energy, the cells may be cultured for a period of time from 12 hours to 24 hours or longer, depending on the cell type. For example, adherent cells typically require a period of settling into the culture well surface. In some examples, the cells are cultured for a period of about 18 hours.

The methods of the present disclosure can also be used to prepare dexosomes (membrane vesicles produced from dendritic cells) or texosomes (membrane vesicles produced by tumour cells). In one example, the dexosomes or texosomes are of human origin. The method according to the disclosure preferably comprises use of an apparatus including a piezoelectric substrate having a working surface, at least one interdigitated transducer located on and in contact with the working surface, and a receptacle located on the working surface for accommodating a population of cells, wherein an alternating signal applied to the interdigitated transducer generates acoustic wave energy through the piezoelectric substrate that can be transferred to the accommodated cells. An example of such an apparatus is described in WO 2019/079857. However, the disclosure is not limited to such a device.

Accordingly, in a second aspect, the present disclosure also provides the use of an apparatus for stimulating the production of EVs from a population of cells, the apparatus comprising:

(i) an acoustic wave generator configured to generate acoustic energy at a selected power and frequency; and

(ii) a receptacle for accommodating a population of cells in a culture media, the receptacle being configured to receive acoustic energy generated by the acoustic wave generator;

wherein the use of the apparatus comprising:

operating the acoustic wave generator to expose cells accommodated in the receptacle to acoustic energy; and

subsequently harvesting the EVs.

In a third aspect, the present disclosure also provides for an apparatus when used according to the methods described herein for stimulating the production of EVs from a population of cells, the apparatus comprising:

(i) an acoustic wave generator configured to generate acoustic energy at a selected power and frequency; and

(ii) a receptacle for accommodating a population of cells in a culture media, the receptacle being configured to receive acoustic energy generated by the acoustic wave generator;

wherein the apparatus is used to expose the cells accommodated in the receptacle to acoustic energy generated by the acoustic wave generator, thereby stimulating the production of EVs from the cells; and

allowing subsequent harvesting of the EVs from the receptacle.

The present disclosure, in a fourth aspect provides an apparatus for use according to the methods described herein in stimulating the production of EVs from a population of cells, the apparatus comprising:

(i) an acoustic wave generator configured to generate acoustic energy at a selected power and frequency; and

(ii) a receptacle for accommodating a population of cells, the receptacle being configured to receive acoustic energy generated by the acoustic wave generator.

The receptacle may define a reservoir configured to accommodate the population of cells in a culture medium. For example, the reservoir may define a well, or in some embodiments, the reservoir may define a channel allowing the cells and culture medium to flow into and/or out of the reservoir.

In some examples, the acoustic wave generator may comprise a piezoelectric substrate, which may define a working surface. The acoustic wave generator may comprise an interdigitated transducer located on and in contact with the working surface of the piezoelectric substrate. When an alternating current electrical signal is applied to the interdigitated transducer, the acoustic energy is generated through the piezoelectric substrate. The acoustic energy may then be transmitted to the population of cells accommodated in the receptacle to promote or stimulate production and release of EVs from the cells. The EVs may then be harvested for use in various applications.

The receptacle may be coupled to the acoustic wave generator. The receptacle may be coupled to the acoustic wave generator with a coupling fluid, such as silicone oil, for example.

The receptacle may be coupled to the working surface of the acoustic wave generator with a coupling material, such as silicone oil, for example, or any other suitable coupling material (e.g., fluid couplant, water, glycerine, or other acoustic transmitting materials including gels and tapes). For liquids and gels, the coupling material may have a viscosity (at 25° C.) in the range of 0.2 cP to 10,000 cP, 1 cP to 1000 cP, 10 cP to 100 cP, 40 cP to 60 cP, or about 50 cP. The coupling material may have a density (at 25° C.) in the range of 0.5 g/ml to 1.5 g/ml, 0.8 g/ml to 1.1 g/ml, or 0.9 g/ml to 1.0 g/ml, for example. In one example, the coupling material has a density in the range of from about 0.95-0.971 g/ml. In a particular example, the coupling material has a density of about 0.963 g/ml.

In some embodiments, the receptacle may be fixed to the acoustic wave generator. In some embodiments, the receptacle may be integrally formed with the acoustic wave generator. In some embodiments, the receptacle may be separate from the acoustic wave generator.

Preferably, the method according to any one of second to fourth aspects is conducted ex vivo.

In one example according the second aspect, the EVs are exosomes as described herein.

In one example, the acoustic wave generator comprises a 500 μm thick 127.86° Y—X rotated lithium niobate (LiNbO3) single-crystal piezoelectric substrate further comprising 40 alternating finger pairs of 11 mm wide and 66 nm thick straight aluminium interdigitated transducer (IDT) electrodes in a basic interleaved configuration. In another example the finger pairs are patterned atop a 33 nm thick chromium adhesion layer. For example, the IDT electrodes may be formed through sputter deposition and standard UV photolithography.

The acoustic wave energy is preferably propagated as a surface acoustic wave (SAW) along the working surface. In one example, the acoustic wave energy is preferably further propagated as a surface reflected bulk wave (SRBW) within the piezoelectric substrate and internally reflected between the working surface and an adjacent surface of the piezoelectric substrate. SRBW can be generated when SAW on the working surface internally reflects between the working and adjacent surface of the piezoelectric substrate. SRBW will therefore be generated at the same frequency as the SAW. Therefore, reference to the application of SAW in the present application can also encompass the application of SRBW when present within the substrate. Further information can be found for example in WO 2016/179664.

In a particular example, the population of cells are exposed to acoustic insonation of about 4 W (input) and about 10 MHz. However, exosome production may be promoted over a range of acoustic energy parameters.

In some examples, the frequency of the applied acoustic energy may be in the range of 7 MHz to 1 GHz, 7 MHz to 100 MHz, 7 MHz to 50 MHz, 7 MHz to 30 MHz, 7 MHz to 20 MHz, 7 MHz to 15 MHz, 7 MHz to 13 MHz, 8 MHz to 12 MHz, 9 MHz to 11 MHz, at least 10 MHz or about 10 MHz, for example.

In some examples, the input power for the acoustic wave generator may be in the range of 0.1 W to 10 W, 1 W to 5 W, 2 W to 4 W, 3 W to 4 W, or about 3.6 W, for example.

In some embodiments, the apparatus may be configured such that the acoustic energy received by the cells and/or cell medium is in the range of about 0.001 W to about 0.1 W, 1 mW to 50 mW, 1 mW to 10 mW, 10 mW to 100 mW, 20 mW to 80 mW, 30 mW to 60 mW, or less than 100 mW, less than 80 mW, less than 50 mW, less than 30 mW, or about 50 mW.

In some examples, the distance between the transducer and the reservoir is in the range of 0 mm (direct contact) to 1 mm.

In some examples, the duration of treatment (or exposure to the acoustic energy) may be in the range of 30 sec to 60 min, 1 min to 60 min, 5 min to 30 min, 10 min to 20 min, 5 min to 15 min, 8 min to 12 min, or about 10 minutes, for example.

In one example, the acoustic insonation is performed at room temperature (i.e 20-25° C.). In one example, the acoustic insonation is performed at about 35-38° C., more preferably about 37° C.

In some embodiments, the cell population may undergo a post exposure incubation. That is, a period of incubation after exposure to the acoustic energy. The post exposure incubation may have a duration in the range of 10 min to 1000 min, 10 min to 100 min, 10 min to 50 min, 20 min to 40 min, 25 min to 35 min, or about 30 minutes, for example.

The inventors have surprisingly found that when the population of cells are recycled through iterative insonation (i.e. acoustic stimulation) and post-excitation incubation steps, production of an homogenous population of exosomes is facilitated thus overcoming some of the drawbacks associated with using exosomes for clinical practice. Moreover, the inventors has also surprisingly found that production yield of exosomes can be achieved through iterative insonation (i.e. acoustic stimulation) and post-excitation incubation steps.

In one example, the cells are exposed to acoustic insonation for a period of time sufficient to generate and release exosomes from the population of cells.

In one example, the population of cells are exposed to the acoustic insonation for a period of about 10 mins followed by a period of 30 mins of which the cells are not exposed to the acoustic insonation but cultured/incubated. More particularly, the cells are incubated at 37° C. between periods of acoustic insonation.

Accordingly, in a further example according to any aspect, the step exposing a cell media comprising the population of cells to an acoustic wave energy comprises exposing the cells to one or more successive periods of acoustic insonation (i.e. acoustic stimulation) followed by incubation in the absence of acoustic insonation.

In one example, the acoustic insonation (i.e. acoustic stimulation) is for a period of about 10 mins followed by an incubation period of about 30 mins.

In one example, the steps of acoustic insonation (i.e. acoustic stimulation) and incubation are repeated at least 4 times. In a further example, the steps of acoustic insonation (i.e. acoustic stimulation) and incubation are repeated between 5 and 20 times, between 8 and 15 times or between 8 and 10 times. Without wishing to be bound by theory, the inventors have found that for stem cells, the optimal number of cycles is 8 and for cancer cells, the optimal number of cycles is 15.

In one example, the steps of acoustic insonation (i.e. acoustic stimulation) and incubation are repeated at least 8 times.

In another example according to any aspect, the method comprises culturing the population of cells in exosome depleted medium prior to acoustic insonation.

In another example according to any aspect, the irradiated cells are incubated for a time period and then the culture medium is collected for harvesting of EVs or exosomes.

In one example, the population of cells is cultured in exosome-depleted medium for about 24-36 hours, preferably about 48 hours. In another example, the population of cells are replenished with exosome-depleted medium immediately prior to acoustic stimulation.

In some examples, the EVs or exosomes are harvested after each cycle of acoustic stimulation.

Advantageously, the inventors have found that cell viability following repeated steps of acoustic insonation (i.e. acoustic stimulation) and incubation is not compromised. Accordingly, the methods of the present disclosure result in the population of cells maintaining a viability of at least 85%, at least 90%, at least 95%, at least 97%, at least 98% or at least 99%. In a further example, cell viability is measured by Trypan Blue exclusion assay. In another example, cell viability is determined by 3-(4,5-dimethylthiazolyl-2)-2,5-diphenyltetrazolium bromide (MTT) proliferation assay. Cell counts may be determined using a cell counter.

Methods for collecting and harvesting EVs or more particularly exosomes from the post-irradiated and cultured cells will be known to persons skilled in the art. One such example includes differential centrifugation. In another example, the method comprises collecting the spent medium from the insonated cells and subjecting the medium to centrifugation and filtration. Further methods for collecting and harvesting include ultra-centrifugation, ultrafiltration, density gradient ultrafiltration, hydrostatic dialysis, size-exclusion chromatography, and precipitation with polymer or protamine. In one example, the centrifugation is carried out at 2,000×g. In a further example, the centrifugation is carried out for a period of time, preferably at least 10 mins. In a further example, the supernatant produced from centrifugation is filtered. In a particular example, the pore size of the filter is in the range of about 0.1 μm to 0.45 μm. Various filter sizes may be contemplated including 0.1 μm, 0.22 μm and 0.45 μm.

In certain examples, the exosomes are isolated/harvested using a commercial kit. Such kits are known in the art and include PureExo® Exosome Isolation Kit, Exosome Isolation kit (Life Technologies, CUSABIO), Exo-Quick (System Bioscience), Exo-Pure 9BioVision, Exo-spin (Cell Guidance System).

In some examples, the steps of centrifugation and isolation/harvesting are performed at 4° C.

In one example, the method according to any aspect results in exosome enrichment of about eight-to-ten fold.

In another example, the method according to any aspect results in an exosome yield in the range of from about 1.7-2.1 fold/hour.

In one example, the exosomes express one or more markers selected from the group consisting of ALIX, TSG101, CD63, syntenin-1, flotillin-1, Rab27a and glyceraldehyde 3-phosphate dehydrogenase (GAPDH). In another example, the exosomes express one or more additional markers selected from CD9, CD81 and HSC70. In another example, the exosomes are negative for calnexin.

In a fifth aspect, the disclosure provides a population of exosomes produced by the method according to any aspect herein. In one example, the exosomes are drug-loaded. In some examples, the population of exosomes may be in the form of a pharmaceutical composition.

In a sixth aspect, the disclosure provides a method for generating drug-loaded exosomes, the method comprising:

(i) delivering a therapeutic agent to a population of cells in culture media;

(ii) exposing the cells to acoustic wave energy; and

(iii) harvesting the exosomes produced from the population of cells following said exposure.

In a seventh aspect, the disclosure provides a method for treating a subject with drug-loaded exosomes, the method comprising:

(i) delivering a therapeutic agent to a population of cells in culture media;

(ii) exposing the cells to acoustic wave energy;

(iii) harvesting the exosomes produced from the population of cells following said exposure; and

(iv) administering the exosomes to the subject.

Methods of delivering a therapeutic agent to cells will be familiar to persons skilled in the art. Such non-limiting examples include transfection, transduction, electroporation or sonoporation.

The therapeutic agent according to the disclosure may be any agent which is capable of treating a disease or disorder in the subject in need thereof. For example, the therapeutic agent may be selected from the group consisting of an siRNA, sdRNA, miRNA, mRNA, antimir, antisense, aptamer, antibody, protein, peptide or small molecule drug or a combination thereof.

The population of cells may be allogeneic or autologous to the subject.

The exosomes may be allogeneic or autologous to the subject.

In some examples, the exosomes as described herein may further comprise a targeting moiety located on the external surface thereof.

### DESCRIPTION OF THE FIGURES

FIG. 1 (a) shows a perspective view and side view schematic of the experimental set up in which the SRBW (not to scale), generated alone a piezoelectric lithium niobate (LiNbO₃) substrate by applying an AC electric signal at the device's resonant frequency (10 MHx) to an interdigitated transducer electrode (IDT) photolithographically patterned on the substrate, is coupled through through a thin layer of silicone oil into a glass-bottom culture plate containing the adherent cells to stimulate their production of exosomes. (b) shows esterase activity isolated from the spent U87-MG cell media as a function of the post-excitation (i.e., after 10 mins of SRBW exposure) incubation time relative to that for the control sample, which comprised unexposed cells incubated over the same period. (c) Enhancement in U87-MG exosome production under the SRBW excitation with increasing input power to the device, indicated by the increase in acetylcholine esterase activity in the spent cell media relative to the unexposed control; the post-excitation incubation period in all cases was fixed at 30 mins. Cell viability data at higher powers beyond 4 W is shown in FIG. 4a. (d) d U87-MG cell viability, as measured from an MTT assay, (e) U87-MG cell population density, and, (f) and (g) relative esterase activity following successive 10 min excitation and 30 min incubation cycles n. The data are represented in terms of the mean value±the standard error over triplicate runs, and the asterisks *** and **** indicate statistically significant differences with p<0:001 and p<0:0001, respectively. The corresponding results for A549 cells is found in found in FIG. 4 and FIG. 5 although the A549 cell esterase activity data for successive cycles is included in panel (f).

FIG. 2 shows the experimental setup (left), which comprises a glass-bottom culture plate containing the cells mounted atop the SRBW device (also shown in top view on the right). A layer of silicone oil is placed in between the device and the glass-bottom culture plate, although this is too thin to be seen.

FIG. 3 shows microscopy images showing the internalisation of exosomes tagged with BODIPY™ TR ceramide in cells counterstained with Hoechst 33342 after (a) 1 hr, (b) 4 hrs and (c) 18 hrs of incubation. The scale bars denote a length of 50 μm. Although exosome uptake in the cells can first be seen after 4 hrs, prolonged incubation led to more significant internalisation, suggesting that overnight post-exposure incubation results in a decrease in the overall exosome quantity.

FIG. 4 shows cell viability following acoustic insonation at various (a) input powers and (b) exposure times; in the former, the exposure time is fixed at 10 mins, whereas in the latter, the power is fixed at 4 W. (c) Relative esterase activity following SRBW exposure at 4 W for 5 and 10 mins compared to that of the unexposed control. The data are represented in terms of the mean value±the standard error over triplicate runs.

FIG. 5 shows (a) Hydrodynamic size of the exosomes isolated from A549 cells. (b) A549 cell viability, as measured from an MTT assay, and, (c) A549 cell population density as a function after successive number n of excitation-incubation cycles. The data are represented in terms of the mean value±the standard error over triplicate runs.

FIG. 6 shows comparison of (a) the number concentration, obtained through NTA, (b) the hydrodynamic size distribution, obtained through DLS, (c) representative cryo-EM images, (d) the protein profile, obtained via Western blotting, and, (e) the band intensities, of EVs isolated from the unexposed control and the SRBW treated U87-MG cells after 7 successive excitation-incubation cycles; the scale bars in (c) represent a length of 50 nm. The data are represented in terms of the mean value±the standard error over triplicate runs, and the asterisks *, ** and *** indicate statistically significant differences with p<0:05, p<0:01 and p<0:001, respectively.

FIG. 7 shows (a) Representative cryo-EM images of the exosomes isolated from U87-MG cells; the scale bars represent a length of 50 nm. (b) TEM images showing significantly more MVBs (circled) in U87-MG cells that were irradiated with the SRBW (right) compared to that in the control (left); the scale bars represent a length of 1 μm.

FIG. 8 shows exosome protein profiling via (a) Western blot analysis, showing the progressive increase in the relative band intensity of b ALIX, c syntenin-1, and, d CD63 with successive number of excitation-incubation cycles, compared to the unexposed control over the same duration. e, f RTqPCR analysis quantifying the mRNA expression of ALIX and CD63 between the unexposed control and the SRBW irradiated cells after successive number of cycles, normalised against GAPDH. The data are represented in terms of the mean value±the standard error over triplicate runs (10 runs for the RT-qPCR experiments), and the asterisks *, ** and *** indicate statistically significant differences with p<0:05, p<0:01 and p<0:001, respectively.

FIG. 9 shows (a) intracellular Ca²⁺ levels, measured from a Fura-2 AM assay, (b) acetylcholine esterase activity, and, (c) mRNA expression of ALIX and CD63, as quantified via RT-qPCR analysis, for cells exposed to the SRBW insonation (i.e. acoustic stimulation) compared to the unexposed control (i), in the absence (−) and presence (+) of extracellular calcium as well as a combination of a Ca²⁺ inhibitor (thapsigargin), ion channel blocker (amiloride HCl) and membrane permeable intracellular Ca²⁺ chelator (BAPTA-AM). The data are represented in terms of the mean value±the standard error over quadruplicate runs, and the asterisks *, **, *** and **** indicate statistically significant differences with p<0:05, p<0:01, p<0:001 and p<0:0001, respectively.

FIG. 10 shows uptake by U87-MG cells of GFP containing exosomes generated from A549 GFP cells counter stained with Hoechst 33342 via the SRBW insonation (i.e. acoustic stimulation). The untreated cells were incubated with exosomes isolated from A549 cells and treated cells were incubated with exosomes isolated from A549 GFP cells. The scale bars denote a length of 50 μm.

### DETAILED DESCRIPTION OF THE DISCLOSURE

### General Techniques and Selected Definitions

The term “and/or”, e.g., “X and/or Y” shall be understood to mean either “X and Y” or “X or Y” and shall be taken to provide explicit support for both meanings or for either meaning.

Reference to the singular forms “a”, “an” and “the” is also understood to imply the inclusion of plural forms unless the context dictates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present invention as described herein can be performed using, unless otherwise indicated, conventional techniques of molecular biology, recombinant DNA technology, cell biology and immunology. Such procedures are described, for example, in Sambrook, Fritsch & Maniatis, Molecular Cloning: A Laboratory Manual, Cold Spring Harbor Laboratories, New York, Second Edition (1989), whole of vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

The term “about”, as used herein when referring to a range is meant to encompass variations of ±20% or ±10%, more preferably ±5%, even more preferably ±1% from the specified amount.

The term “drug” as used herein is intended to refer to an active pharmaceutical ingredient (API) defined by the WHO as any substance of combination of substances used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings.

The term “exosome” as used herein refers to an extracellular vesicle that is released from a cell upon fusion of an intermediate endocytic compartment, the mutivesicular body (MVB), with the plasma membrane of the cell. This liberates intraluminal vesicles (ILVs) into the extracellular milieu and the vesicles thereby released are referred to as exosomes.

The term extracellular vesicle (EV) is understood to refer to particles that are naturally released from a cell. EVs are described herein are intended to refer to exosomes, microvesicles (ectosomes) and microparticles. The term is also intended to include particles having a size range between 30 nm and 1000 nm, more preferably 30-500 nm, even more preferably 30-200 nm.

The term “microvesicle” (also known as an ectosome) as used herein refers to a membrane bound vesicle containing phospholipids surrounding a small amount of cytosol and ranging from 100 nm to 1000 nm which are shed from almost all cell types, including megakaryocytes, blood platelets, monocytes, and neutrophils. They are released into the extracellular environment by the outward budding and fission of the plasma membrane.

The term “pharmaceutical composition” refers to a composition which is, within the scope of sound medical judgement, suitable for use in contact with the tissues of human beings and animals without excessive toxicity, irritation, allergic response or other problems or complications commensurate with a reasonable benefit/risk ratio.

As used herein, the term “treat” or “treatment” or “treating” shall be understood to refer to the medical management of a patient with the intent to cure, ameliorate, stabilize, or prevent a disease, pathological condition or disorder. This term includes active treatment, i.e. treatment directed specifically toward the improvement of a disease, pathological condition, or disorder. In addition, this term includes palliative treatment, i.e. treatment designed for the relief of symptoms rather than curing the disease, pathological condition or disorder; and supportive treatment, i.e. treatment employed to supplement another specific therapy directed towards the improvement of the associated disease, pathological condition or disorder.

As used herein an “antibody” refers to natural or synthetic antibodies that selectively bind to a target antigen. The term includes polyclonal and monoclonal antibodies. In addition to intact immunoglobulin molecules, also included are “fragments or polymers of those immunoglobulin molecules, and human or humanized versions of immunoglobulin molecules that selectively bind the target antigen.

The term “peptide” refers to a natural or synthetic molecule comprising two or more amino acids linked by the carboxyl group of one amino acid to the alpha amino group of another.

As used herein, the term “subject” shall be taken to mean any subject, including a human or non-human subject. The non-human subject may include non-human primates, ungulate (bovines, porcines, ovines, caprines, equines, buffalo and bison), canine, feline, lagomorph (rabbits, hares and pikas), rodent (mouse, rat, guinea pig, hamster and gerbil), avian, and fish. In one example, the subject is a human.

### Extracellular Vesicles

Extracellular vesicles are a class of membrane-bound organelles secreted by various cell types. They include exosomes which are 30-150 nm diameter membranous vesicles of endocytic origin, microvesicles (also referred to as ectosomes) which are 50-1000 nm diameter that are shed directly from the plasma membrane and apoptotic bodies or blebs which are 50-5000 nm diameter released by dying cells. For the purpose of the present disclosure, an extracellular vesicle is understood to refer to an exosome or endosome.

In a particular example, extracellular vesicles (EVs) comprise a population of vesicles, such as where about 80-90% or more of the vesicles are exosomes. Exosomes are typically 30-150 nm vesicles, preferably 20-120 nm vesicles, responsible for the transport of a myriad of molecular cargo including protein, lipids, mRNA and miRNA. Receptors on the cell surface also play a role in intracellular communication. Exosomes are composed of a lipid membrane bilayer structure that expresses the surface ligands and receptors from source cells. The lipid membrane bilayer structure is typically rich in lipid rafts which are subdomains of the plasma membrane that contain high concentrations of cholesterol and glycosphingolipids. This property gives the exosomes their rigidity.

Technologies used for the detection of particles like exosomes and microvesicles include electron microscopy, flow cytometry, dynamic light scattering (DLS), and nanoparticle tracking analysis (NTA). Each method mentioned above has advantages and drawbacks when used to detect extracellular vesicles. Electron microscopy can directly show that vesicles exist in a sample, but cannot provide quantitative data, and the fixation process can alter vesicle shape and size. Flow cytometry is typically limited to the identification of particles greater than 300 nm, preventing the detection of smaller microvesicles and all exosomes. Recently developed flow cytometry protocols have lowered this limit, but the detection of sub 100 nm particles, like exosomes, still remains an outstanding problem (van der Vlist EJ, Nolte-'t Hoen ENM, Stoorvogel W, Arkesteijn GJA, Wauben MHM. (2012) Nat Protoc.; 7:1311-1326). Challenges of detecting extracellular vesicles with DLS include: 1) the low refractive index of vesicles, and 2) a bias towards detection of larger particles when used with heterogeneous solutions. This makes it problematic to distinguish between microvesicles (>100 nm) and exosomes (<100 nm) in mixed solution. NTA is perhaps the most promising method because it can identify both microvesicles and exosomes and is not dependent on the refractive index of the vesicles. However, without a fluorescently labeled antibody directed towards a vesicle surface marker, or without use of a vesicle isolation method to reduce polydispersity of the sample, there can be considerable intra-assay count variability (see Kastelowitz N et al (2014) Chembiochem 15(7):923-928 for review). Both DLS and NTA rely on the relationship between particle size and diffusion coefficient to determine the size of the extracellular vesicles in solution. For example, going from a vesicle 30 nm in diameter to 130 nm in diameter changes the diffusion coefficient by ˜12 μm2/s, but from 900 nm to 1000 nm the diffusion coefficient changes by only ˜0.05 μm2/s.

In addition to fundamental properties such as diffusion coefficient, exosomes and microvesicles also display surface markers that can be used for quantification and detection. Universal markers commonly used to identify exosomes are better characterized, and include transmembrane proteins like tetraspanins (CD9, CD63, CD81, and CD82) and MHC class I and II, and cytosolic proteins like heat shock proteins (HSP-70 and HSP-90). Source specific markers that represent the proteome of the cell of origin can also be used for exosome identification.

Detection of extracellular vesicle proteins is relatively straight forward using analytical techniques like western blot or ELISA. Exosomes also express internal markers such as Alix and Tsg101. Additional markers expressed by exosomes include syntenin-1, flotillin-1 and Rab27a.

Membrane curvature sensing peptides are a unique class of molecules that can sense the physical state of the exosome membrane. Curvature sensing peptides such as myristoylated alanine-rich C kinase substrate (MARCKS) and C2BL3C, is a 12 amino acid long peptide that was cyclized using “Click” chemistry, as well as BAR domains, the ALPS motif of ArfGAP1, or α-synuclein can be utilized.

The exosome or microvesicle according to the present disclosure may be derived from an exosome producing cell selected from, but not limited to immune cells such as B cells, T lymphocytes, dendritic cells (DCs) and mast cells. The exosome may also be selected from an exosome producing cell selected from glioma cells, platelets, reticulocytes, neurons, intestinal epithelial cells and tumour cells and tumour cell lines. Because exosomes are produced naturally from cells they are tolerated by humans and animals and have very little immunogenicity. Accordingly, due to their low immunogenicity, the exosomes or microvesicles may be autologous or allogeneic. For example, the exosomes can also be obtained from any autologous patient-derived, heterologous haplotype-matched or heterologous stem cells so to reduce or avoid the generation of an immune response in a patient to whom the exosomes are delivered. Any exosome-producing cell can be used for this purpose. In the case of autologous exosomes, the exosomes may be generated from a sample of the tumour which is to be treated. In the case of allogeneic exosomes, they may be derived from a cancer cell line.

Exosomes or microvesicles for use in the disclosed compositions and methods can be derived from any suitable cell, including the cells identified above. Exosomes have also been isolated from physiological fluids, such as plasma, urine, amniotic fluid and malignant effusions. Non-limiting examples of suitable exosome producing cells for mass production include dendritic cells (e.g., immature dendritic cell), Human Embryonic Kidney 293 (HEK) cells, 293T cells, Chinese hamster ovary (CHO) cells, colorectal cancer (HT-29), human breast cancer (MDA-MB-231), human ESC-derived mesenchymal stem cells, A549 cells and U87-MG cells.

In some examples, the exosome is a tumour exosome. In a further example, the exosome comprises one or more tumour associated antigens specific to the tumour from which the exosome is derived.

In some examples, the exosome is derived from a fruit or plant. Examples of such exosomes are described in Ju S et al., (2013) Mol Ther 21:1345-57.

In some examples, the exosome is derived from milk. Examples of such exosomes are described in Zempleni J, (2017) Genes Nutr. 2017; 12:12.

In some examples, the exosomes are derived from dendritic cells (DCs), such as immature DCs. Exosomes produced from immature DCs do not express MHC-II, MHC-I or CD86. As such these exosomes do not stimulate naïve T cell to a significant extent and are unable to induce a response in a mixed lymphocyte reaction.

Exosomes produced from cells can be collected from the culture medium by any suitable method. Typically, a preparation of exosomes can be prepared from cell culture or tissue supernatant by centrifugation, filtration or combinations of these methods. For example, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 μm filter), gradient ultracentrifugation (for example, with sucrose gradient) or a combination of these methods.

Exosome yield released from the cells can be measured according to art known methods, for example by measuring acetylcholine esterase activity. Exosome concentration and/or particle size can measured by nanoparticle tracking analysis based on light scattering.

Exosome morphology may be examined by microscopic techniques such as transmission electron microscopy and cryo-electron microscopy as described in the examples herein. Other methods include scanning electron microscopy (SEM) or focused ion beam (FIB-SEM). A review of these methods can be found in Drasler B et al., (2017) Nanomedicine 12(10):1095.

### Exosome Labelling

In certain examples, exosomes may be labelled with a diagnostic agent or detectable agent.

In certain examples, the exosomes may be coupled to a detectable label to facilitate detection. Examples of detectable labels include various enzymes, prosthetic groups, fluorescent materials, luminescent materials, bioluminescent materials, electron dense labels, labels for MRI, and radioactive materials. Examples of suitable enzymes include horseradish peroxidise, alkaline phosphatise, β-galactosidase, or acetylcholinesterase. Examples of suitable prosthetic group complexes include streptavidin/biotin and avidin/biotin. Examples of suitable fluorescent materials include umbellifone, fluorescein isothiocyanate, rhodamine, dischlorotriazinylamine fluorescein, dansyl chloride or phycoerythrin. An example of a luminescent material includes luminol. Examples of bioluminescent materials include luciferase, luciferin, and aequorin, and examples of suitable radioactive material include ¹²⁵I, ¹³¹I, ³⁵S, ¹⁸F, ⁶⁴Cu, ^{94m}Tc, ¹²⁴I, ¹¹C, ¹³N, ¹⁵O, ⁶⁸Ga, ⁸⁶Y, ⁸²Rb or ³H.

In vivo and in vitro tracking of exosomes can be achieved by various methodologies. For example, Lai et al., (2014) ACS Nano 8:483-94 have engineered human embryonic kidney 293T exosomes expressing a membrane-bound Gaussian luciferase fused to a biotin receptor domain and then complexed with biotin expressed on the exosomes with fluorescent Alexa-Fluor 680-streptavidin, thus allowing the exosomes to be tracked in vivo either by bioluminescence or fluorescence. In addition, Oosthuyzen W et al., (2013) J Physiol 591:5833-42 have used fluorophore-conjugated antibodies against exosomal proteins CD24 and aquaporin 2 (AQP2) to identify a subpopulation of CD24- and AQP2-positive exosomes by using nanoparticle tracking analysis (NTA) in vitro. Alternatively, anti-CD63 antibody conjugated microbeads and secondary antibody-conjugated Q-dots can be used to track exosomes using NTA (Wang J et al., (2016) Stem Cells Int 2639728.

### Therapeutic Agents

The present disclosure also contemplates drug-loaded exosomes which exosomes can be produced according to the methods described herein. Drug-loaded exosomes comprise a therapeutic agent internalised within the exosome. In some examples, the therapeutic agent is delivered to the population of cells and the drug becomes internalised within the exosomes produced and secreted by the cells. In another example, the therapeutic agent is introduced into the exosomes after they have been produced and secreted from the cells according to the methods described herein.

The therapeutic agent according to the disclosure may be selected from the group consisting of chemotherapeutics, hormones, analgesics, anti-inflammatory agents, anti-bacterial agents, anti-coagulants, anti-hypertensive agents, antibodies, antibody conjugates, proteins, biologically active proteins, fusion proteins, peptides, polypeptides (e.g. cytokine, chemokine, enzyme, hormone etc.), vaccine antigens, blood products, anti-toxins, polynucleotides, and small molecules and combinations of any of the foregoing.

### Administration of Exosomes to a Subject

The exosomes produced by a method of the disclosure may be provided in the form of a composition comprising a pharmaceutically acceptable carrier and/or excipient. The choice of excipient or other elements of the composition can be adapted in accordance with the route and device used for administration.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Administration to a subject (e.g. human) is preferably by injection. The administration route may be intramuscular or intravascular (e.g. intravenous), intracerebral, subcutaneous or transdermal. A physician will be able to determine the required route of administration for each particular patient.

A composition of the invention may be formulated for parenteral, intramuscular, intracerebral, intravascular (including intravenous), subcutaneous or transdermal administration.

Suitable carriers for the present disclosure include those conventionally used, e.g. water, saline, aqueous dextrose, lactose, Ringer's solution a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

The present disclosure also provides medical uses and methods for treatment in vitro and in vivo of the exosomes produced herein. In one example, the drug-loaded exosomes are used in cancer treatment. In a further example, the cancer is selected from the group consisting of solid tumours, including for example fibrosarcoma, myxosarcoma, liposarcoma, chondrosarcoma, osteogenic sarcoma, chordoma, angiosarcoma, endotheliosarcoma, lymphangiosarcoma, synovioma, lymphangioendotheliosarcoma, mesothelioma, Ewing's tumour, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumour, cervical cancer, testicular tumour, lung carcinoma, small cell lung carcinoma, bladder carcinoma, epithelial carcinoma, glioma, astrocytoma, melanoma, brain cancer and retinoblastoma.

Additional disorders that may be treated include viral infection, neurological disorders, rheumatic diseases, immunological disorders, or bacterial infection.

### Apparatus

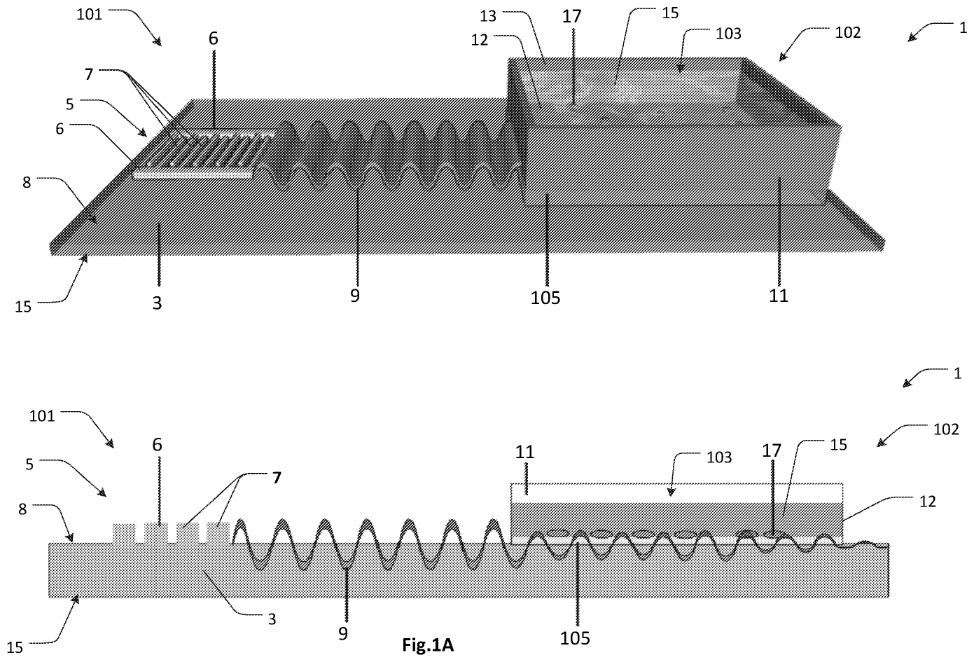

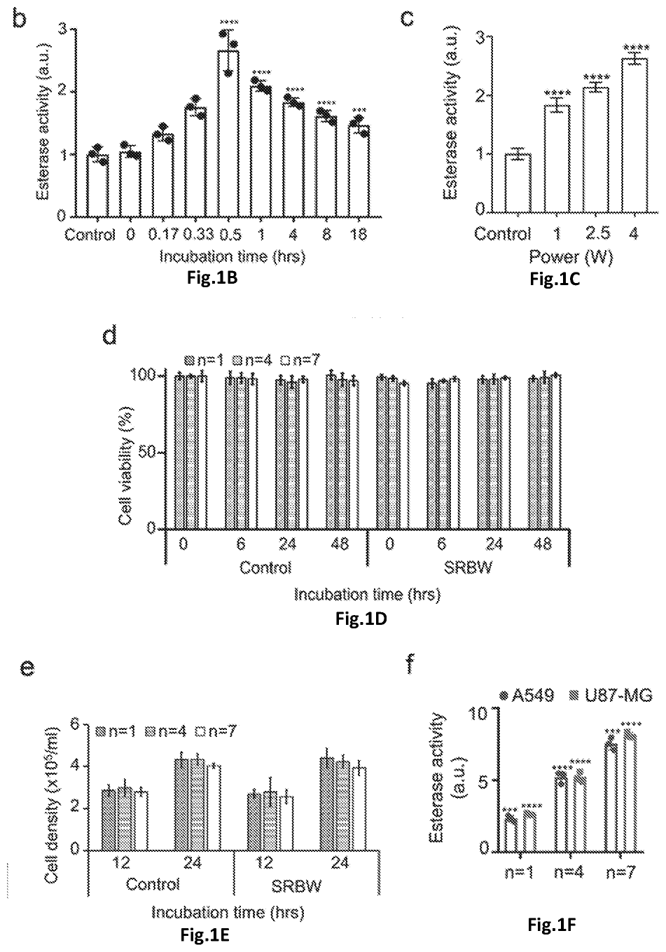

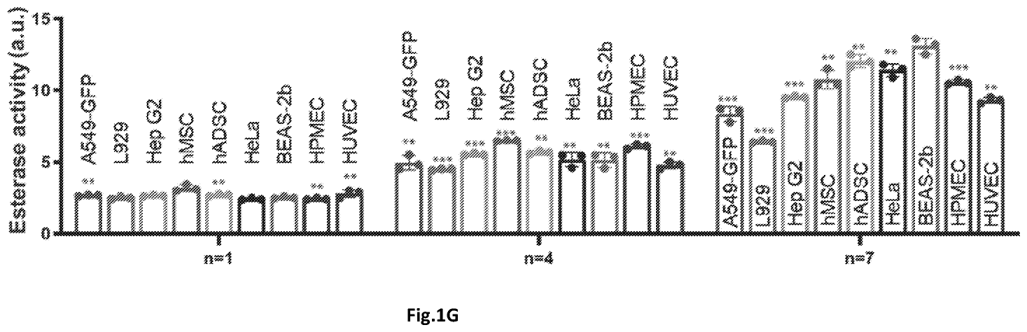

Referring to FIG. 1a, an apparatus 1 for insonating a population of cells to acoustic energy (i.e., acoustic waves or vibrations) is shown, according to some embodiments.

The apparatus 1 comprises an acoustic wave generator 101 configured to generate acoustic energy at a selected power and frequency; and a receptacle 102 for accommodating a population of cells. The receptacle 102 is configured to receive acoustic energy generated by the acoustic wave generator 101.

The acoustic wave generator 101 shown in FIG. 1a comprises a piezoelectric element. However, in other embodiments, the acoustic wave generator 101 may comprise any other suitable device for generating acoustic energy or vibrations, including speakers, vibrators or other electromechanical devices.

The receptacle 102 may define a reservoir 103 configured to accommodate the population of cells in a cell medium. For example, the reservoir 103 may define a well, or in some embodiments, the reservoir may define a channel allowing the cells and culture medium to flow into and/or out of the reservoir.

The receptacle 102 may be coupled to the acoustic wave generator 101. For example, the receptacle 102 may be coupled to the acoustic wave generator 101 with a coupling material 105 to facilitate transmission of the acoustic energy from the acoustic wave generator 101 to the receptacle 102. For example, the coupling material 105 may comprise a fluid couplant, such as silicone oil.

In some embodiments, the receptacle 102 may be fixed to the acoustic wave generator 101. For example by adhesive bonding or by mechanical fastening.

In some embodiments, the receptacle 102 may be separate from the acoustic wave generator 101. For example, the receptacle 102 may not be in direct contact with the acoustic wave generator 101 during operation. The acoustic energy may be transmitted from the acoustic wave generator 101 to the receptacle 102 via a transmission medium.

The apparatus 1 shown in FIG. 1a includes a piezoelectric substrate 3, for example, lithium niobate (LiNbO₃), defining a working surface 8 on which electrodes 6 of an interdigitated transducer (IDT) 5 are photolithographically patterned. The width of and gaps between the IDT fingers 7 of the electrodes 6 determine the resonant wavelength and resonant frequency of the acoustic wave generator 101.

Applying an alternating electrical signal to the IDT electrodes 6 at this resonant frequency with the aid of a signal generator and amplifier (not shown) then generates surface acoustic waves (SAW) 9 that propagate as Rayleigh waves along the working surface 8 of the substrate 3 upon which the IDT electrodes 6 are positioned. In addition to the SAW 9, surface reflected bulk waves (SRBW) can also propagate internally within the substrate 3 between the working surface 8, and an adjacent opposing surface 15 of the substrate 3. The SRBW is internally reflected between the working surface 8 and the opposing surface 15 and preferably also provides acoustic wave energy to the receptacle 102. The propagation of the SRBW may be enhanced by configuring the substrate 3 so that it has a thickness that is approximately equal to the SAW wavelength. Further description of SRBWs can be found in International Publication No. WO2016/179664 (RMIT University).

The receptacle 102 of the apparatus 1 of FIG. 1a is shown in the form of a well plate 11, comprising a base 12 and side walls 13 made from glass or other acoustically transmitting materials such as acrylic. The receptacle 102 is disposed on the working surface 8 of the substrate 3. The receptacle 102 defines multiple wells each configured to accommodate a population of cells 17 in cell media 15. Alternatively, the receptacle 102 may comprise one or more petri dishes, transwell culture plates, microarray plates, cell flack, or other standard laboratory items for cell culture made from glass or other suitable materials. Additionally, the receptacle 102 may also comprise a fluid channel or conduit as part of a flow through system.

In some embodiments, the receptacle 102 may be integrally formed with the acoustic wave generator 101. For example, the receptacle 102 may comprise a portion of the acoustic wave generator 101, such as a portion of the substrate 3. The reservoir 103 may be defined by a recess in the working surface 8, for example.

It is also envisaged that a receptacle 102 having side walls only and no base wall could be used so that the cells and media 15 can be in direct contact with (i.e., directly coupled to) the working surface 8.

The receptacle 102 may be positioned on the work surface 8 to transmit the acoustic wave energy of the SAW 9 and preferably SRBW to the accommodated cells 17. A thin layer of silicone oil (or another fluid couplant, including water, glycerine, or other acoustic transmitting materials including gels and tapes) may be placed between the working surface 8 and base wall 12 of the well plate 11 to facilitate the coupling between the acoustic wave generator 101 and the receptacle 102, and to facilitate the transmission of the acoustic wave energy into the wells. The silicone oil may also mitigate or reduce any acoustic impedance mismatch.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Materials

Unless otherwise specified, sodium chloride, methanol, ethanol, isopropanol, liquid ethane, RNasefree water, nuclease-free water, glycerol, glycerine, non-fat skimmed milk, silicone oil, dimethylsulfoxide (DMSO), sodium cacodylate buffer, uranylacetate, β-mercaptoethanol, Tween® 20, sodium dodecyl sulphate (SDS), Trizma® (Tris) base, phosphate buffered saline (PBS), amiloride hydrochloride (HCl), Tris-HCl, chloroform, ammonium persulphate (APS), bis-acrylamide, 1,2-bis(2-aminophenoxy)ethaneN,N,N′,N′-tetraacetic acid (BAPTA-AM), Gibco penicillin-streptomycin, acetylthiocholine, 5,5′ dithiobis(2-nitrobenzoic acid), ethylenediaminetetraacetic acid (EDTA), trypsin-EDTA, paraformaldehyde, glutaraldehyde, osmium tetroxide, potassium ferrocyanide, Triton™ X-100, thapsigargin, bovine serum albumin (BSA), fetal bovine serum (FBS), Roswell Park Memorial Institute (RPMI) 1640 medium, Dulbecco's Modified Eagle Medium (D-MEM) without calcium, Dulbecco's phosphate buffered saline (D-PBS), bromophenolblue, radioimmunoprecipitation (RIPA) assay buffer, biotinylated protein ladder, Hoechst 33342, Trypan Blue solution, Fura-2 acetoxymethyl ester (AM), 3-(4,5-dimethylthiazol-2-yl)-2,5-diphenyltetrazolium bromide (MTT), BODIPY™ TR ceramide, Vybrant® MTT cell proliferation assay kit, bicinchoninic acid (BCA) protein assay kit, PureExo® exosome isolation kit (101 Bio LLC, Mountain View, Calif., USA), LunaScript® RT SuperMix kit, Luna® Universal qPCR Master Mix, TRiZOL™ reagent, Pierce™ ECLWestern blotting detection reagent, nitrocellulose membrane (0.45 m), protease inhibitor cocktail tablet, polyacrylamide gel, Formvar/carbon coated and holey carbon grids (Emgrid Pty. Ltd., Gulfview Heights, SA, Australia), A549 and U87-MG cells (American Type Culture Collection, Manassas, Va., USA), Exosome Spin Columns (MW 3000), T25 cell culture flask, MatTek 24-well glass-bottom plates and Nunc™ Lab-Tek™ II Chamber Slide and Chambered Coverglass were acquired from Thermo Fischer Scientific Pty. Ltd. (Scoresby, VIC, Australia).

Anti-GAPDH mouse antibody, anti-ALIX mouse antibody, anti-Rab 27a rabbit antibody, antimouse horse radish peroxidase (HRP) conjugated antibody, anti-biotin HRP linked antibody and anti-rabbit HRP-conjugated antibody was obtained from Cell Signaling Technology Inc. (Danvers, Mass., USA), anti-TSG101 mouse antibody and anti-syntenin-1 rabbit antibody from Thermo Fisher Scientific Pty. Ltd. (Scoresby, VIC, Australia), anti-calnexin rabbit antibody from Abcam (Cambridge, UK), anti-flotillin-1 mouse antibody from BD Biosciences (San Jose, Calif., USA) and anti-CD63 mouse antibody from Invitrogen (Carlsbad, Calif., USA).

The following primers used for RT-qPCR analysis were acquired from Integrated DNA Technologies Inc. (Coralville, Iowa, USA):

ALIX (forward):
5′-GACGCTCCTGAGATATTATGATCAGA-3′,
ALIX (reverse):
5′-ACACACAGCTCTTTTCATATCCTAAGC-3′,
CD63 (forward):
5′-TAGATTCGGCAGCCATGGCGGTGGAA-3′
CD63 (reverse):
5′-ACTGACCAGACCCCTACATCACC-3′,
GAPDH(forward):
5′-CATGTTCCAATATGATTCCACC-3′,
GAPDH(reverse):
5′-GATGGGATTTCCATTGATGAC-3′.

### Device Fabrication

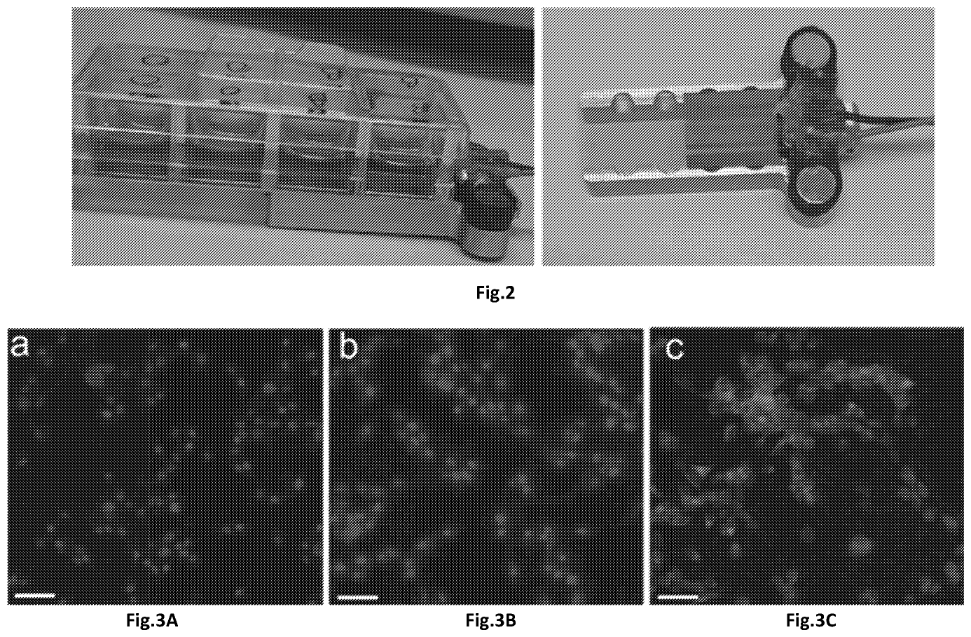

The SRBW devices, schematically illustrated in FIG. 1a and shown in the images in FIG. 2, comprised 500 μm-thick 127.86° Y—X rotated lithium niobate (LiNbO3) single-crystal piezoelectric substrates (Roditi Ltd., London, UK) on which 40 alternating finger pairs of 11-mm-wide and 66-nm-thick straight aluminium interdigitated transducer (IDT) electrodes in a basic full width interleaved configuration were patterned atop a 33-nm-thick chromium adhesion layer through sputter deposition and standard UV photolithography. The width and the gap of the IDT fingers (λ/4) then sets the SRBW wavelength λ=398 μm and hence the device's resonant frequency f=10 MHz. To generate the SRBW, an alternating electrical signal is applied to the IDTs at the resonant frequency using a signal generator (SML01, Rhode & Schwarz Pty. Ltd., North Ryde, NSW, Australia) and amplifier (10 W1000C, Amplifier Research, Souderton, Pa., USA). As depicted in FIG. 1a, a thin layer of silicone oil with viscosity 45-55 cP and density 0.963 g/ml at 25° was sandwiched between the SRBW device and the glass-bottom chamber slide in which the cells were contained to aid coupling of the acoustic energy from the device into the wells.

### Cell Culture and Acoustic Exposure

U87-MG human glioblastoma cells and A549 adenocarcinomic human alveolar basal epithelial cells were respectively cultured in DMEM and RPMI medium supplemented with 10% FBS and 1% penicillin-streptomycin (100 units/ml) in a humidified incubator maintained at 37° C. and 5% CO2. The cell media for the batches referred to in FIG. 1G are as follows:

A549-GFP, L929, HeLa—Rosewell Park Memorial institute 1640 (RPMI);

Hep G2, hMSC, hADSC—Dulbecco's modified eagle's media (DMEM with low glucose);

BEAS-2b—Bronchial epithelial growth medium (BEGM);
HPMEC—Microendothelial growth medium (EGM-2MV);
HUVEC—Macroendothelial growth medium (EGM2).

The cells were grown in a standard T25 flask until they reached 80-90% confluency, following which they were detached using 0.05% trypsin-EDTA 24 hrs prior to the experiments, reseeded in the 8-well plates at a density of 300,000 cells per well and incubated for 18 hrs. The cells were then thrice-washed with PBS and replenished with exosome-depleted medium (DMEM with 1% penicillin-streptomycin and 10% exosome-depleted FBS, the latter prepared by centrifuging at 121,800×g for 19 hrs from which the supernatant was filtered using a 0.22 μm filter and used immediately or stored) for 48 hrs; this washing and replenishing step was also repeated immediately prior to the experiment. The cells in the well plate were then irradiated with the SRBW at the prescribed input power to the device for the stipulated duration. Following cessation of the acoustic field, the cells and media were incubated at 37° C. for the prescribed time period, following which the spent culture media was immediately collected and the exosomes isolated for further characterisation. For the control, the cells were seeded at same the density and incubated over the same time period.

### Cell Viability and Proliferation

The viability of cells following their exposure to the SRBW insonation (i.e. acoustic stimulation) was assessed using a MTT proliferation assay in which the treated cells were washed with PBS immediately after collecting the spent media following which 3-(4,5-dimethylthiazolyl-2)-2,5-diphenyltetrazolium bromide (MTT) solution at a final concentration of 0.5 mg/ml in serum-free medium was added to each well and incubated for 3 hrs. The absorption of formazan crystals dissolved in DMSO was measured at 570 nm using a spectrophotometric plate reader (CLARIOstar®, BMG LabTech, Mornington, VIC, Australia) and normalised with respect to the absorbance of the control containing cells at the same concentration that were not exposed to the SRBW insonation (i.e. acoustic stimulation) but incubated for the same time period. The viability of the SRBW treated cells after 6, 24 and 48 hrs was also analysed to determine long term cytotoxicity effects. The cells' ability to continue to proliferate following exposure to the SRBW insonation (i.e. acoustic stimulation), on the other hand, was evaluated using a Trypan Blue exclusion assay in which the SRBW treated cells were trypsinized immediately and reseeded. The cell count was then determined after 24 and 48 hrs using Trypan Blue (0.4%) solution with a cell counter (Invitrogen Countess™, Thermo Fisher Scientific Pty. Ltd., Scoresby, VIC, Australia).

### Exosome Isolation, Quantification and Characterisation

The spent medium, collected from the SRBW-treated and control (untreated) samples following the stipulated excitation and subsequent incubation period, was centrifuged at 2,000×g for 15 mins at 4° C., from which the supernatant was collected and filtered using the PureExo® exosome isolation kit. The isolated exosomes were stored at 4° C. for a week or at −80° C. for up to three months. Total exosomal protein content was estimated using BCA analysis in which 5 μl of the exosome isolate was reconstituted in PBS to 150 μl and mixed with BCA reagent at a 1:1 volume ratio prior to incubation at 37° C. for 2 hrs, after which the solution was brought to room temperature and its absorbance was measured at 562 nm with a spectrophotometric plate reader (CLARIOstar®, BMG LabTech, Mornington, VIC, Australia).

The exosomes released were quantified by measuring their acetylcholine esterase activity. Briefly, 25 μl of the exosome isolate was suspended in PBS (pH 8) and incubated in 1.25 mM acetylthiocholine and 0.1 mM 5,5′ dithiobis(2-nitrobenzoic acid) at 37° C., following which the change in solution absorbance at 412 nm was continuously monitored over 1 hr using a spectrophotometric plate reader (CLARIOstar®, BMG LabTech, Mornington, VIC, Australia). The concentration of the exosomes was also evaluated using nanoparticle tracking analysis (NTA; NanoSight NS300 and NTA 3.2 software, Malvern Panalytical Ltd., Malvern, UK) whereas their size distribution was evaluated from dynamic light scattering measurements (DLS; Zetasizer Nano S, Malvern Instruments Ltd, Malvern, UK) at an emission wavelength of 658 nm.

Additionally, the morphology of the isolated exosomes was visually examined via transmission electron microscopy (TEM; 1010, JEOL, Frenchs Forest, NSW, Australia) and cryo-electron microscopy (cryo-EM; Tecnai F30; FEI, Eindhoven, Netherlands). For TEM, 4 μl of isolated exosomes in PBS were adsorbed onto activated Formvar/carbon coated grids for 10 mins followed by incubation in 10 μl 1% uranylacetate for 1 min. The grids were then washed twice in MilliQ® water (18.2 M·cm, Merck Millipore, Bayswater, VIC, Australia) and left overnight to dry. For cryo-EM, a 3 μl aliquot of the purified exosome sample was added onto a holey carbon grid, blotted and plunge-frozen into pre-cooled liquid ethane. Imaging was carried out at an accelerating voltage of 200 kV.

To visualise the MVBs within the cells, cell samples were quickly removed following the experiments and fixed in paraformaldehyde/glutaraldehyde followed by 1% osmium tetroxide and 1.5% potassium ferrocyanide. The fixed cells were then subjected to ethanol dehydration and infiltrated into resin, after which they were sectioned using an ultramicrotome and stained for visualisation under the TEM.

Exosome tracing studies were conducted by tagging the isolated exosomes with BODIPY™ TR ceramide (final dye concentration of 10 μM). Following incubation for 20 mins at 37° C., excess dye was removed using Exosome Spin Columns (MW 3000). The exosomes were then added to unstained recipient cells and incubated for different periods (1, 4 and 18 hrs), after which the media containing the tagged exosomes was removed. The cells were subsequently washed thrice in PBS and fixed with 4% paraformaldehyde prior to imaging (EVOS M5000, Life Technologies Corp., Bothell, Wash., USA). Hoechst 33342 was used as the counterstain.

### Protein and Gene Profiling

Exosome marker (ALIX, TSG101, CD63, syntenin-1, flotillin-1, Rab27a and GAPDH) and negative marker (calnexin) proteins were identified from exosomes isolated from both the control and irradiated samples using Western blot analysis. 60 μg of the isolated exosomes was lysed in RIPA buffer, which was then denatured in reducing SDS loading buffer (62.5 mMTris-HCl (pH 6.8), 2% SDS, 25% glycerol, 0.01% bromophenol blue and freshly added 5% β-mercaptoethanol) by heating at 95° C. for 5 mins. The samples were then run on a 10% polyacrylamide gel and transferred onto a nitrocellulose membrane at 60 mV for 60 mins. The nitrocellulose membrane was blocked for 1 hr in 5% non-fat skimmed milk in Tris buffered saline solution (TBST; 20 mM Tris, 150 mM sodium chloride, 0.05% Tween® 20) and incubated overnight in the antibody (primary antibody at 1:1000 dilution, anti-mouse antibody at 1:30,000 dilution and anti-rabbit antibody at 1:50,000 dilution) at 4° C., following which the membranes were treated with the appropriate HRP-conjugated secondary antibody in 0.05% TBST at 37° C. for 1 hr. The membranes were then visualised in a gel imager (LI-COR Biotechnology, Lincoln, Nebr., USA) following incubation in Pierce™ ECL Western blotting detection reagent at room temperature for 2 mins. For CD63, non-reducing conditions were employed in which the exosome lysate was mixed with SDS loading buffer but in the absence of β-mercaptoethanol.

To measure total RNA content, the control and SRBW treated cells were homogenised using TRiZOL™ and chloroform, and centrifuged at 1200×g to obtain an RNA-containing aqueous layer and a DNA- and protein-containing layer. The RNA was then precipitated with isopropanol and washed in ethanol, dissolved in RNAse-free water with 0.1 μM EDTA and quantified using a UV spectrophotometer (NanoDrop™ One; Thermo Fisher Scientific, Waltham, Mass., USA). cDNA was synthesized with the LunaScript® RT SuperMix kit and RT-qPCR carried out using the Luna® Universal qPCR Master Mix with the aforementioned primers.

### Calcium Influx Studies

The cells were seeded at a density of 0:05×10⁶ cells per well in a 24-well glass-bottom plate and incubated at overnight in a humidified incubator. After incubation, they were treated with combinations of amiloride HCl (100 μM, for 60 mins)—a Ca²⁺ ion channel blocker, thapsigargin (100 nM, for 25 mins)—an endoplasmic reticulum calcium ion inhibitor, and, BAPTA-AM (10 μM, for 25 mins)—a membrane permeable calcium chelator that removes intracellular calcium, following which they were incubated in the presence of 10 μM Fura2-AM for 60 mins at 37° C. to measure the intracellular Ca²⁺ levels. The cells were then washed to remove the extracellular dye and replenished with DMEM, taking care to protect them from exposure to light. The media devoid of calcium from the inhibitor-treated cells was subsequently replaced with media containing calcium, and, where appropriate, exposed to the SRBW insonation (i.e. acoustic stimulation). Changes in the fluorescence intensity were measured with a spectrophotometric plate reader (CLARIOstar®, BMG LabTech, Mornington, VIC, Australia).

### Statistical Analysis

Data presented in this study are expressed as the mean±the standard error of replicate measurements, and analysed using a two-tailed, unpaired Student's t-test, where applicable.

### Example 1 Quantification of Exosome Presence and Exosome Secretion

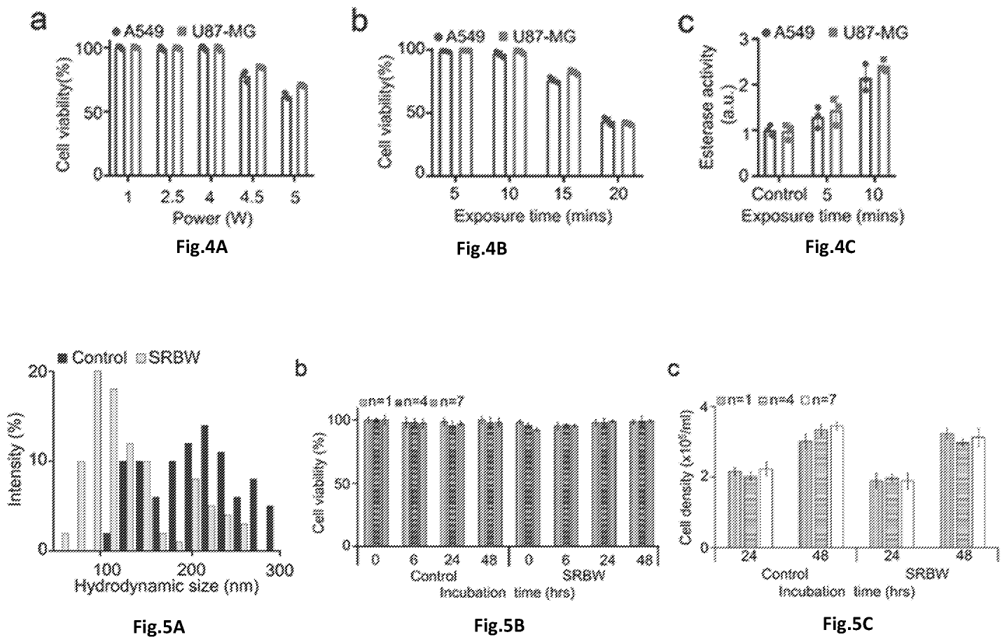

FIG. 1b shows the acetylcholine esterase activity that quantifies the presence of U87-MG exosomes in the sample as a function of the post-excitation incubation period following 10 mins of Shamble Bandwidth (SRBW) excitation using the setup shown in FIG. 1a and FIG. 2; parenthetically, the inventors note that although the esterase activity, in general, is not just specific to exosomes but rather to all extracellular vesicles (EVs), these measurements were carried out after the exosomes were purified from the EVs in the spent media using an exosome isolation kit and hence the esterase activity in this case can be considered as a positive marker for the exosomes. It can be seen that the esterase activity increased significantly by approximately 1.7-fold in the first 30 mins following application of the SRBW insonation for 10 mins to the cells, suggesting elevated levels of exosomes that were secreted by the cells within this period, after which the number of exosomes gradually reduced with increasing incubation time, possibly due to their internalisation by neighbouring cells (Maia, J et al., (2018) Frontiers in Cell and Developmental Biology 6:18.; Gonda, A et al., (2019) Molecular Cancer Research 17:337-347) as evident from the images in FIG. 3 that show their uptake over 18 hrs. Increasing the input power to the device and hence the SRBW excitation amplitude, on the other hand, can be seen in FIG. 1c to increase exosome production although we note that increasing the power beyond approximately 4 W (as well as the exposure time beyond 10 mins) leads to a reduction in the cell viability (FIGS. 4a and b). As such, the post-excitation incubation period was therefore fixed at 30 mins and the input power to the device at 4 W in all subsequent experiments to maximise both exosome production and cell viability.

### Example 2 Viability of Cells Post Excitation

FIGS. 1d and 1e (see n=1 data) showed that the majority of U87-MG cells (approximately 95%) remained viable and adherent following SRBW insonation and continued to proliferate normally, consistent with results from preceding studies employing similar high frequency acoustic forcing for intracellular macromolecular uptake (Ramesan, S et al., (2018) Nanoscale 10:13165-13178). Similar results were also observed for A549 cells (FIG. 5 b and c). Unlike low frequency ultrasound (10-100 kHz up to 1 MHz) typically used in sonoporation, the considerably higher frequencies and significantly lower powers (one to two orders of magnitude) associated with the SRBW excitation or its surface acoustic wave (SAW) counterpart do not generate any appreciable cavitation (Rezk, A. R., et al (2020) The Journal of Physical Chemistry Letters (11) 4655-4661) to induce pore formation in the cell plasma membrane, which is known to inflict considerable damage to the cell. Rather, it was postulated in Ramesan S et al., that the high frequency excitation was only sufficient to drive reversible permeabilisation of the membrane by inducing transient structural reorganisation of the lipids that make up the plasma membrane (Ramesan S et al., and Reusch, T et al., (2014) Physical Review Letters 113:118102) which immediately reseals upon relaxation of the acoustic signal. This would not just explain the high viabilities observed in the present work but also suggests the possibility that the acoustic excitation could also be responsible for enhancing the secretion of the exosomes produced under the same stimuli.

Moreover, the high cell viability offers the unique possibility for further increasing the exosome yield from the same cell population by repeatedly exposing the same batch of cells to successive excitation-incubation cycles, each cycle n comprising SRBW insonation for 10 mins followed by a 30 min incubation period. As shown in FIG. 1f, four- and eight-fold increases in the relative esterase activity after n=4 and n=7 cycles, corresponding to a total duration of 160 and 280 mins, respectively, were observed without any appreciable effects on the cell homeostasis, i.e., no significant decreases could be seen in the viability of the cells (FIG. 1d and FIG. 5b) or their ability to proliferate (FIGS. 1e and 5c) compared to the untreated cells over the same period. The inventors note the possibility of some exosomes being trapped in the membranes during their isolation (Ramesan, S., et al. (2018) Nanoscale 10:13165-13178) and hence the likelihood that the number of exosomes produced could be higher in each successive cycle. Moreover, given that the cell viabilities are maintained even after 7 cycles, it is possible to continue the excitation-incubation cycles to further increase the exosome yield—such an ability to recycle the cells constitutes a significant advantage over other methods, both in terms of maintaining proteome and lipidome homogeneity in the exosome population, which is highly desirable and a significant challenge at present for exosome therapeutics (Ferguson, S. W., et al. (2016) Journal of Controlled Release 228:179-190 and Willis, G. R., et al. (2017) Frontiers in Cardiovascular Medicine 4:63) and in reducing the cost of the cell feedstock, which can be considerable, particularly for large-scale exosome manufacture (Colao I. L., (2018) Trends in Molecular Medicine 24:242-256.

Referring to FIG. 1G, further batches of cells were exposed to successive excitation-incubation cycles, including: A549-GFP (human male lung carcinoma stably expressing green fluorescent protein (GFP)); L929 (mouse fibroblast cells); Hep G2 (human hepatocellular carcinoma); hMSC (human mesenchymal stem cells); hADSC (human adipose derived stem cells); HeLa (human adenocarcinoma); Beas-2b (human bronchial epithelial cells); HPMEC (human pulmonary microendothelial cells); HUVEC (human umbilical vascular endothelial cells). Each successive excitation-incubation cycle n comprised SRBW insonation for 10 mins followed by a 30 min incubation period. It is clear from FIG. 1G that esterase activity increases with the number of cycles for all tested cell types by varying amounts.

### Example 3 Physical Characteristics of the Exosomes

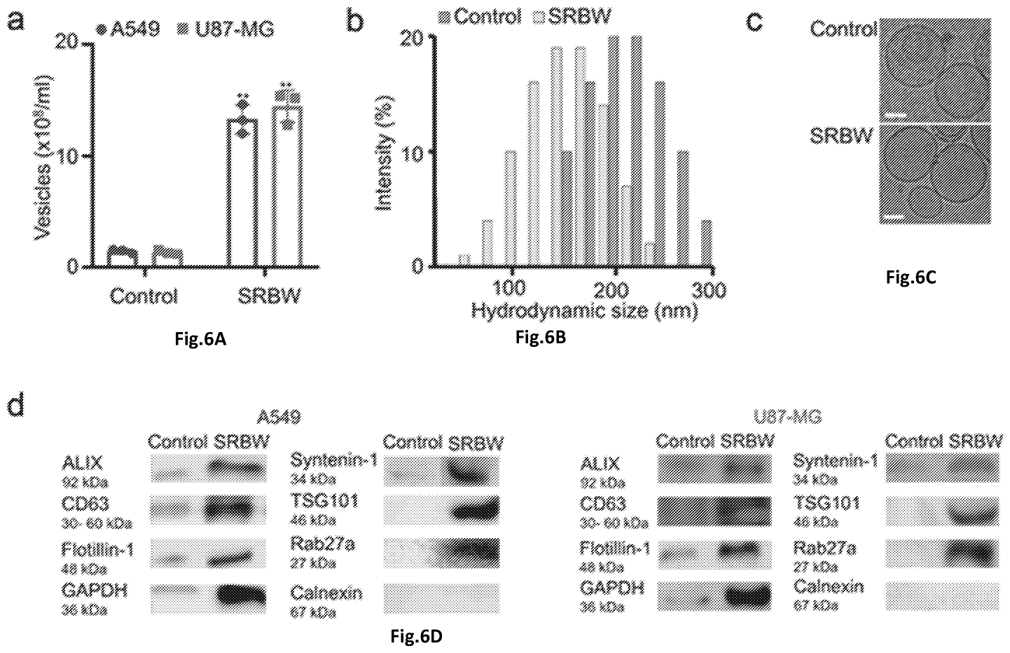

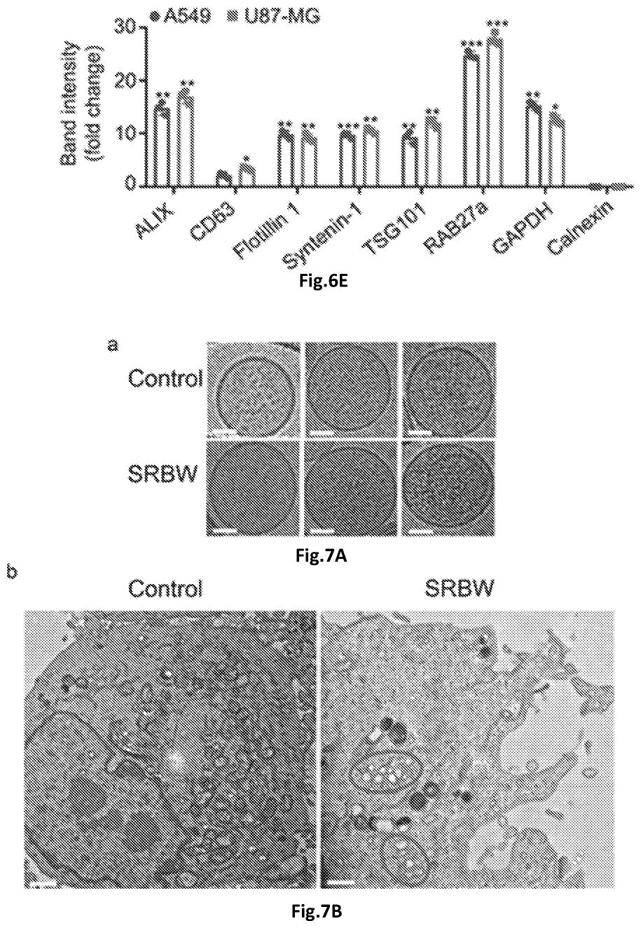

The number concentration and size distribution of the exosomes isolated from the control and irradiated cells after n=7 cycles, obtained via nanoparticle tracking analysis (NTA) and dynamic light scattering (DLS), respectively, are shown in FIGS. 6a and b, respectively (the size distribution for exosomes isolated from A549 cells can be found in FIG. 5a). While the concentration verifies the enrichment (approximately ten-fold; cf. eight-fold enrichment obtained through quantification with the esterase activity in FIG. 1f) in the exosome production obtained from the iterative excitation-incubation steps, a comparison of the sizes of the exosomes between the control and irradiated samples not only shows slightly smaller hydrodynamic diameters, but also indicates that a large proportion of the EVs that were produced under SRBW insonation appear to consist primarily of exosomes, which, by definition, have size ranges between 30 and 150 nm. Taken together with the sphericity of the entities observed in the cryo-electron microscopy (cryo-EM) images in (FIG. 6c, which are representative across all of the results obtained (see also FIG. 7a), this suggests minimal, if not negligible, formation of apoptotic bodies, which are usually irregular in dimension. Moreover, it is noted from the cryo-EM images that the membrane integrity of the exosomes obtained following SRBW insonation appears to be preserved.

However, as physical characterisation does not completely rule out the existence of most other classes of EVs such as microvesicles and apoptotic bodies, the inventors looked to evidence beyond physical characterisation, in particular exosomal protein profiling via Western blotting, which may allude to the mechanism by which exosomes are generated, and, in doing so, verify the existence of the exosomes produced through acoustic stimulation. FIGS. 6d and 6e reveal an abundance of proteins in the exosome lysate after n=7 successive excitation-incubation cycles that are implicated in exosome biogenesis following SRBW exposure, specifically those involved in the endosomal sorting complexes required for transport (ESCRT) pathway that orchestrates the generation of late endosomes, i.e., multivesicle bodies (MVBs) within the cell, whose fusion with the plasma membrane leads to the release of intraluminal vesicles into the extracellular matrix as exosomes (the TEM images of fixed cells following acoustic excitation in FIG. 7b shows an increase in MVBs within the cells compared to the control).

### Example 4 Exosome Protein Profiling and Gene Expression

In particular, the inventors note the overexpression of ALIX (ALG-2 (apoptosis-linked gene 2)-interacting protein X) and TSG101 (Tumour Suppressor Gene 101)—the two accessory proteins involved in the ESCRT machinery, and CD63—which has been reported to also be present in the ESCRT pathway, in addition to that of other exosomal markers, namely, flotillin-1—essential for membrane invagination which is a precursor to MVB formation, syntenin-1—a cargo sorting protein without which exosomes cannot be generated, and Rab27a—which facilitates MVB docking onto the plasma membrane and whose elevated levels do not just imply an enhancement of exosome production in the cell but also an increase in their secretion from the cell (Keller S et al., (2006) Immunology Letters 107:102-108; Abels E. R., (2016) Cellular and Molecular Neurobiology 36:301-312). Correspondingly, the inventors note the absence of calnexin—an endoplasmic reticulum marker that constitutes a negative control in exosome production (Martins, T. S et al., PLoS One 2018, 13, e0198820)—that also confirms the absence of microvesicles (Haraszti, R. A. et al., (2016) Journal of Extracellular Vesicles 5:32570) and apoptotic bodies in the irradiated sample (Tucher, C. et al., (2018) Frontiers in Immunology 9:534.; and Lötvall, J. et al., (2014) Journal of Extracellular Vesicles 3:26913).

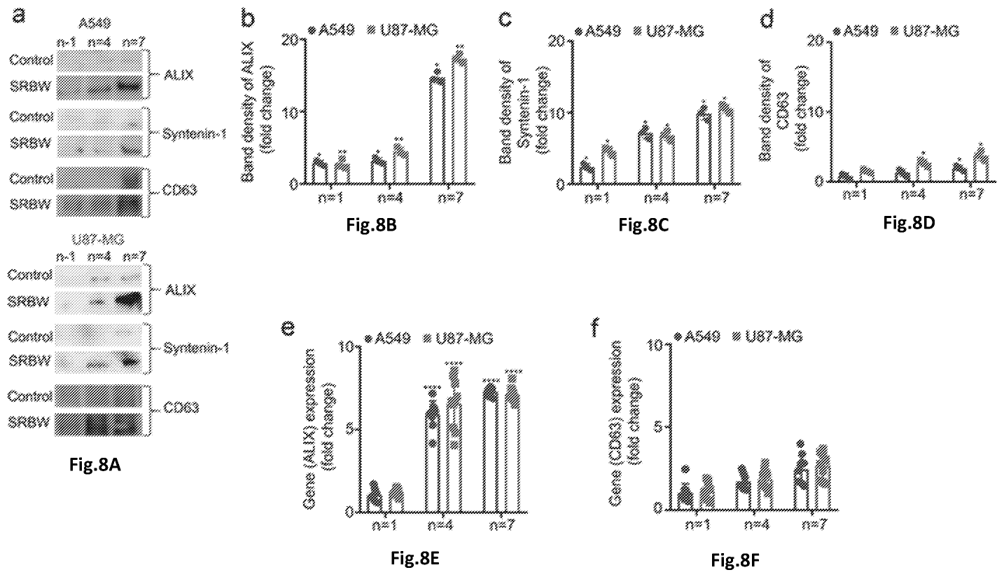

Moreover, the exosomal protein expression can also be seen to increase with the number of excitation-incubation cycles (FIG. 8). In particular, the inventors observed the ALIX (FIG. 8b) and syntenin-1 (FIG. 8c) levels to increase progressively with successive cycling, whereas the CD63 (FIG. 8d) level can be seen to increase initially before plateauing between n=4 and n=7, consistent with the mRNA expression levels in FIGS. 8e and 8f, measured using real-time quantitative reverse transcription polymerase chain reaction (RT-qPCR) with GAPDH as a housekeeping gene. This suggests that the SRBW irradiated cells might favour an ALIX-mediated pathway in which the ESCRT accessory component ALIX complexes with syntenin-1 to regulate its role in membrane recruitment and intraluminal budding (Friand, V. et al., (2015) Biology of the Cell 107:331-341); the relatively lower increase (approximately three-fold) in CD63 expression compared to that for ALIX (approximately six-fold) is likely because the affinity of CD63 to syntenin-1 is roughly ten times less than that of ALIX (Baietti, M. F., (20112) Nature Cell Biology 14:677-685).

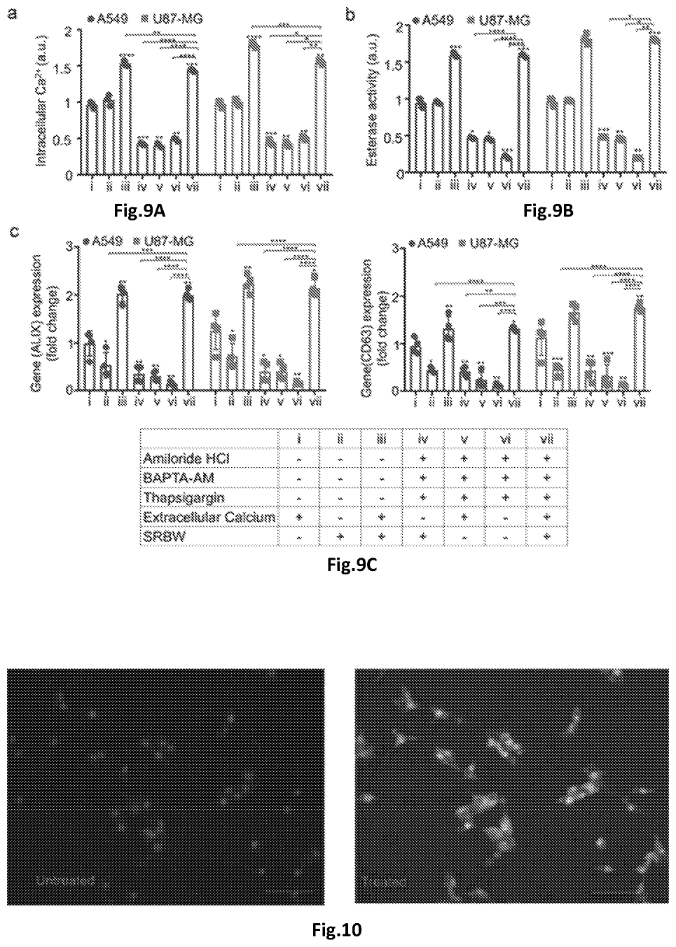

The role of the acoustic stimuli in the overexpression of exosomal proteins can be understood from intracellular calcium profiling since intracellular calcium plays a crucial role in ESCRT recruitment and hence endosomal release. More specifically, it has been reported that cells under stress are typically associated with increased calcium ion (Ca²⁺) levels, either due to its release from the intracellular Ca²⁺ store or through its uptake into the cell from the extracellular milieu. That the acoustic stimulation increases intracellular Ca²⁺ through the latter mechanism, i.e., internalisation of Ca²⁺ from the extracellular milieu, is evident from the measurements of the intracellular Ca²⁺ level in FIG. 9a, which shows an elevated reading (iii) for the SRBW irradiated sample above the baseline level associated with its unexposed counterpart (i). This is further verified in the case when no Ca²⁺ was present in the extracellular milieu (ii), in which case no significant change in the intracellular Ca²⁺ level compared to the control (i) was observed even when the cells were exposed to the acoustic insonation.

A similar increase in the intracellular Ca²⁺ level for the acoustically irradiated cells (vii) can be seen even in the presence of calcium channel blockers amiloride HCl and thapsigargin and a membrane permeable intracellular Ca²⁺ chelator BAPTA-AM, which act to deplete the intracellular calcium store (iv, v, vi). The inventors note that such an increase in the intracellular Ca²⁺ was also observed when cells are exposed to similar high frequency vibrational excitation, and is likely due to the increase in membrane permeability as a consequence of the transient reorganisation of the plasma membrane lipid structure during high frequency acoustic stimulation. Similar trends can be seen in the acetylcholine esterase activity (FIG. 9b) and the mRNA overexpression associated with ALIX and CD63 (FIG. 9c) wherein the inventors observed across-the-board enhancement in exosome production with SRBW insonation even in the presence of the inhibitor(s) and/or chelator, thus highlighting the essential role of intracellular Ca²⁺ in producing transcriptomic changes under the SRBW stimuli.

Taken together, these results suggest that the increase in intracellular Ca²⁺ uptake into the cell in response to high frequency stimulation has a two-fold effect. In addition to directly enhancing intracellular transport across the plasma membrane due to its permeabilisation as a consequence of the acoustically-driven vibrational stressing of the membrane, the immediate healing of the membrane upon relaxation of the SRBW excitation, given the transient and reversible nature of this process involving rearrangement of its lipid structure, also leads to recruitment of extracellular Ca²⁺ This then prompts recruitment of ESCRT nucleating factors ALIX and TSG 101, which forms a complex with syntenin-1 at the site of repair, consistent with previous studies where ALIX- and CD63-positive vesicle release was observed in response to Ca²⁺ triggering following membrane puncture (Scheffer, L. L. et al., (2014) Nature Communications 5:5646. The requirement for Ca²⁺ to be present in the extracellular environment for its internalisation into the cell to trigger ESCRT recruitment and endosomal release, suggests the SRBW insonation does not induce endoplasmic reticulum (ER) stress to trigger the release Ca²⁺ from the internal cellular store. That the high frequency acoustic insonation (i.e. acoustic stimulation) is capable of altering cellular activity without imparting ER stress, as confirmed by the absence of calnexin—an ER stress marker—in the protein profile of the isolated EVs in FIG. 6e, is unique and quite unlike other techniques for enhancing exosomal yield that involve application of external stimuli to the cell.

### Example 5 Preparation of Diva-Loaded Exosomes

A549GFP is a stable A549 cell line transfected with GFP protein. The exosomes isolated from the cells were able to transfect U87 cells but GFP in exosomes were not detected in the spectrophotometer.

The A549-GFP (commercially available) are a stable cell line which has EGFR protein tagged with GFP. The exosomes were isolated from normal A549 cells and A549-GFP cells. The exosomes isolated were then incubated with U87 cells (4 Hrs). Lack of green fluorescence in U87 cells treated with A549 exosomes (control) compared to that of U87 cells with exosomes from A549-GFP cells shows that the exosomes produced from acoustically triggered cells could be used for delivering therapeutics as well.

### REMARKS

The studies herein show that low level insults involving high frequency acoustic stimulation to mammalian cells enhances production of EVs through a calcium-dependent ALIX-mediated pathway whilst maintaining very high (95%) cell viability. In addition to showing that the EVs that are generated primarily consists of exosomes, the inventors elucidate via protein and calcium profiling the mechanism by which such enhancement in exosome production transpires. In particular, the gentle vibration of the cells at high frequencies drives transient reorganisation of the lipid structure of the plasma membrane that increases its permeability without inflicting significant damage (e.g., via poration) to it. This augmentation in membrane permeability, together with its healing when the acoustic signal is relaxed, promotes recruitment of calcium ions into the cell, initiating the assembly of ESCRT accessory proteins at the site of repair, which, in turn, orchestrates the cascade of events—MVB fusion, intraluminal vesicle accumulation and cargo release—that lead to the production of exosomes.

Through an iterative procedure in which the cells are repeatedly exposed to cycles of acoustic insonation (i.e. acoustic stimulation) for 10 mins followed by 30 mins of post-excitation incubation, it was shown that it is possible to obtain an eight- to ten-fold amplification in the number of exosomes in just 7 cycles corresponding to a total treatment duration of 280 mins, which is equivalent to an approximate yield of 1.7-2.1 fold/hr. This scalable (through massive parallelisation, given the low cost of the SRBW devices (around US$1/device), achieved by exploiting the economies-of-scale through mass nanofabrication (Rezk, A. R. et al., (2018) Lab on a Chip 18:406-411) platform thus offers a facile means by which the current bottlenecks in exosome technology (namely, the inability to sufficiently produce the large amounts required that are sufficiently homogeneous from the same cell source for clinical use) can be circumvented, therefore offering a potential solution that enables the exciting promise of exosomes for diagnostics and therapeutics to be realised.

1. A method for stimulating the production of extracellular vesicles (EVs) from a population of cells, the method comprising:
(i) exposing a culture media comprising the population of cells to acoustic wave energy and
(ii) harvesting the EVs produced from the population of cells following the exposure.

2. The method according to claim 1, wherein the EVs are exosomes, microvesicles (ectosomes), or oncosomes or a combination thereof.

3. The method according to claim 1 or 2, wherein the EVs comprise greater than 80% exosomes.

4. The method according to any one of claims 1 to 3, wherein the exosomes have a hydrodynamic diameter size of between 30-150 nm.

5. The method according to any one of claims 1 to 4, comprising the further step of preparing the population of cells.

6. The method according to any one of claims 1 to 5, wherein the cells are cultured for at least 12 hours prior to exposure to acoustic wave energy.

7. Use of an apparatus for stimulating the production of EVs from a population of cells, the apparatus comprising:
(i) an acoustic wave generator configured to generate acoustic energy at a selected power and frequency; and
(ii) a receptacle for accommodating a population of cells in a culture media, the receptacle being configured to receive acoustic energy generated by the acoustic wave generator;
wherein the use of the apparatus comprises:
operating the acoustic wave generator to expose cells accommodated in the receptacle to acoustic energy; and
subsequently harvesting the EVs.

8. An apparatus for use in stimulating the production of EVs from a population of cells according to the method of any one of claims 1 to 6, the apparatus comprising:
(i) an acoustic wave generator configured to generate acoustic energy at a selected power and frequency; and
(ii) a receptacle for accommodating a population of cells in a culture media, the receptacle being configured to receive acoustic energy generated by the acoustic wave generator.

9. The method, use or apparatus according to any one of claims 1 to 8, wherein the receptacle defines a reservoir configured to accommodate the population of cells in a culture medium.

10. The method, use or apparatus according to any one of claims 1 to 9, wherein the acoustic wave generator comprises a piezoelectric substrate defining a working surface and an interdigitated transducer located on and in contact with the working surface of the piezoelectric substrate.

11. The method, use or apparatus according to any one of claims 1 to 10, wherein the receptacle is coupled to the acoustic wave generator with a coupling material.

12. The method, use or apparatus according to any one of claims 1 to 11, wherein the acoustic wave energy is propagated as a surface acoustic wave (SAW) along the working surface.

13. The method, use or apparatus according to any one of claims 1 to 11, wherein the acoustic wave energy is propagated as a surface reflected bulk wave (SRBW) within the piezoelectric substrate and internally reflected between the working surface and an adjacent surface of the piezoelectric substrate.

14. The method, use or apparatus according to any one of claims 1 to 13, wherein the frequency of the applied acoustic energy is in the range of about 7 MHz to about 1 GHz.

15. The method, use or apparatus according to any one of claims 1 to 14, wherein the input power for the acoustic wave generator is in the range of about 0.1 W to about 10 W.

16. The method, use or apparatus according to any one of claims 1 to 15, wherein the exposure of the population of cells to the acoustic energy is in the range of about 30 sec to about 60 min.

17. The method, use or apparatus according to any one of claims 1 to 16, wherein the step of exposing a culture media comprising the population of cells to an acoustic wave energy comprises exposing the cells to one or more successive periods of acoustic insonation followed by incubation in the absence of acoustic stimulation.

18. The method, use or apparatus according to claim 17, wherein the steps of acoustic insonation and incubation are repeated at least 7 times.

19. The method, use or apparatus according to claim 18 wherein the EVs are harvested after each cycle of acoustic stimulation.

20. The method, use or apparatus according to any one of claims 2 to 19, wherein the exosomes express ALIX but are negative for calnexin.

21. A population of exosomes produced by the method according to any one of claims 1 to 6.

22. A method for generating drug-loaded exosomes, the method comprising:
(i) delivering a therapeutic agent to a population of cells in culture media;
(ii) exposing the cells to acoustic wave energy; and
(iii) harvesting the exosomes produced from the population of cells following said exposure.

23. A method for treating a subject with drug-loaded exosomes, the method comprising:
(i) delivering a therapeutic agent to a population of cells in culture media;
(ii) exposing the cells to acoustic wave energy;
(iii) harvesting the exosomes produced from the population of cells following said exposure; and
(iv) administering the exosomes to the subject.
